SEQUENCE LISTING (2 sequences)

SEQ ID NO: 1 - protein, 1073 aa, synthetic construct
MMKVIWFSSLICLVIQCGGDTGPIICAGPIHSNKSADIPHLLGYSEKICQIDRLIHVSSWLRNHSQFQGYVGQRGGRSQVSYYPAENSYSRWSGLLSPCDADWLGMLVVKKAKGSDMIVPGPSYKGKVFFERPTFDGYVGWGCGSGKSRTESGELCSSDSGTSSGLLPSDRVLWIGDVACQPMTPIPEETFLELKSFSQSEFPDICKIDGIVFNQCEGESLPQPFDVAWMDVGHSHKIIMREHKTKWVQESSSKDFVCYKEGTGPCSESEEKTCKTSGSCRGDMQFCKVAGCEHGEEASEAKCRCSLVHKPGEVVVSYGGMRVRPKCYGFSRMMATLEVNEPEQRIGQCTGCHLECISGGVRLITLTSELKSATVCASHFCSSATSGKKSTEIQFHSGSLVGKTAIHVKGALVDGTEFTFEGSCMFPDGCDAVDCTFCREFLKNPQCYPAKKWLFIIIVILLGYAGLMLLTNVLKAIGVWGSWVIAPVKLVFAIIKKLMRAVSCLMGKLMDRGRQVIHEEIGENREGNQDDVRIEMARPRRVRHWMYSPVILTILAMGLAEGCDEMVHADSKLVSCRQGSGNMKECVTTGRALLPAVNPGQEACLHFTAPGSPDSKCLKIKVKRINLKCKKSSSYFVPDARSRCTSVRRCRWAGDCQSGCPPHFTSNSFSDDWAGKMDRAGLGFSGCSDGCGGAACGCFNAAPSCIFWRKWVENPHGIIWKVSPCAAWVPSAVIELTMPSGEVRTFHPMSGIPTQVFKGVSVTYLGSDMEVSGLTDLCEIEELKSKKLALAPCNQAGMGVVGKVGEIQCSSEESARTIKKDGCIWNADLVGIELRVDDAVCYSKITSVEAVANYSAIPTTIGGLRFERSHDSQGKISGSPLDITAIRGSFSINYRGLRLSLSEITATCTGEVTNVSGCYSCMTGAKVSIKLHSSKNSTAHVRCKGDETAFSVLEGVHSYSVSLSFDHAVVDEQCQLNCGGHESQVTLRGNLIFLDVPKFVDGSYMQTYHSTVPTGANIPSPTDWLNALFGNGLSRWILGVIGVLLGGLALFFLIMSLFKLGTKQVFRSRTKLA

SEQ ID NO: 2 - DNA, 3378 bp, synthetic construct
  source 1..3378: mol_type = other DNA
acacagagacggccaacaatgatgaaagtcatctggttctcctctctgatctgcttagtcattcaatgcggtggggatacaggcccaatcatctgcgcaggacccatccactcaaacaagagtgctgacataccccacctgcttggctactctgagaagatttgtcagatagatcggctgatacatgtttcgtcatggcttagaaaccactcacaatttcagggctacgtagggcagcgaggtggacgatctcaggtgagctactacccagctgaaaattcttactcaaggtggagtggacttctaagcccctgtgatgctgattggcttgggatgcttgtcgtgaagaaggccaaggggtctgacatgatagttccagggccttcatacaaggggaaagtcttttttgaacggccaacttttgatggatacgtaggctggggctgtggcagtgggaagtctaggactgagtcaggtgagctctgcagttcagactccgggactagttctggtcttctgccctcagatagggttctctggataggtgatgttgcttgtcagccaatgacacccatccctgaggagacatttctggagctgaagagctttagccaaagtgaattcccagacatatgcaaaattgatggcattgtgttcaaccagtgtgagggtgagagtctacctcagccctttgatgttgcatggatggatgttggccactctcataaaatcatcatgagggagcacaagaccaaatgggtacaagagagctcatccaaggattttgtgtgctacaaggaagggactggaccttgttctgaatcagaagaaaagacttgcaagaccagtggatcatgcaggggggacatgcagttttgcaaggtggcaggttgtgaacatggggaagaggcatctgaagccaagtgcagatgctcacttgtgcacaagcccggggaagtcgttgtgtcatatggagggatgcgtgtcagaccaaagtgctatggtttctccagaatgatggcaacactggaggtgaacgaaccagagcagaggattggtcaatgcactggctgccatctagaatgcataagtgggggtgtgaggctaatcactctaaccagtgagctcaagtcagctactgtctgcgcttctcacttctgtagttctgccacaagtggtaagaaaagcacggagattcaattccactcaggatcattggttgggaaaacagcaatccacgtcaaaggggcattggtagatggaactgaattcacatttgagggtagttgcatgttcccagatggttgtgatgcagtggactgcacattctgtcgtgagtttctaaaaaatccccagtgctaccctgcaaagaagtggctgttcatcattattgtcatcctccttggatatgcaggcctcatgctactcaccaatgttcttaaggcaattggggtttggggatcatgggtcatagctccagtgaagctagtgtttgccatcataaagaaactaatgagagctgtgagctgcctgatggggaaattgatggatagaggaaggcaagtgatccatgaggaaataggggagaatagagagggcaaccaagatgatgttaggattgagatggccagacccaggagggtaaggcattggatgtactcgcctgtcatcctgactattctagcaatggggcttgctgagggctgtgatgagatggtccatgctgattctaaacttgtttcgtgcaggcaagggagcggaaatatgaaggaatgtgtcacaactgggagggcgcttcttcctgcagtgaacccagggcaagaggcgtgtctgcacttcacggcacctggaagtccggactcaaaatgtctcaaaattaaggttaagaggatcaacctaaaatgtaaaaagtcatcatcatattttgttcctgatgcccggtccaggtgtacatcagtgaggagatgtcgttgggcaggggactgccagtctgggtgcccccctcatttcacatccaactccttttctgatgattgggcaggtaagatggacagggctggtctaggattcagtggctgctctgatggatgtggaggagcagcctgtggttgctttaatgcggccccttcttgcattttctggaggaaatgggtagagaacccacatgggatcatctggaaagtatctccatgtgccgcatgggtcccatcagcggttatagagctaacaatgccctcaggggaggtgaggacattccaccccatgagcggaatccccacacaagtcttcaagggtgtgagtgtaacttacttgggctcagatatggaggtgtcaggcttgactgacctgtgtgagatagaagagctcaagtcaaagaagcttgctttagctccctgcaaccaggctggtatgggggttgtaggcaaggttggagagatacagtgcagtagcgaggaaagtgcccgtaccataaaaaaagatgggtgcatatggaatgcagaccttgtgggtatagagctacgggtggatgacgctgtgtgctactctaagatcactagtgtggaggcagttgcaaactactccgccatacccaccactattggggggctgaggtttgagagaagccatgacagccagggcaaaatatctggtagccccctggacatcacagctataagaggatctttttctatcaattatagaggccttcgactgagcctctcagaaattactgctacttgcacaggggaagtgacaaatgtgagtgggtgttattcttgcatgacaggcgccaaagtctccatcaagttgcatagcagcaaaaatagcactgcccatgtaagatgcaaaggggatgagactgcattcagtgtcttggagggagtccatagctactctgtcagtctcagttttgatcatgcagtagttgatgagcagtgccaactgaactgtgggggacatgagagccaagtgactctgagaggcaacctcatcttcctggatgtcccgaaatttgtggatggcagctacatgcagacatatcatagtactgtgcccacaggggcaaatatcccaagccctacagattggctaaatgccttgtttggcaatgggctgagtaggtggatcctgggggtgataggggtcctactggggggattggctctctttttcttgatcatgtctttgttcaagctgggaacaaaacaggtatttcgatcgaggacgaagctggcttaaatgggcatatttctggtctaatgaccctctgggggcagtgctctcaggggagttggctcactgtgtgcatgttttgtggtcctggctcacattcttagagcatagaggttctattgaagtgttggccggtctttgtgt

(19) United States
(12) Patent Application Publication
(10) Pub. No.: US 2025/0339507 A1
(43) Pub. Date: Nov. 6, 2025

(54) MRNA VACCINE FOR BANDAVIRUS DABIEENSE AND PREPARATION METHOD THEREOF

(71) Applicants: NANJING MEDICAL UNIVERSITY, Nanjing City (CN); Pharmaceutical Industry Technology Research Institute of Nanjing Medical University, Taizhou City (CN)

(72) Inventors: Xinjian LIU, Nanjing City (CN); Minjun JI, Nanjing City (CN); Jin ZHU, Nanjing City, (CN); Tingting ZHOU, Nanjing City (CN); Zihan WU, Nanjing City (CN); Yuting WEN, Nanjing City (CN); Yuchen YANG, Nanjing City (CN)

(21) Appl. No.: 19/271,871

(22) Filed: Jul. 17, 2025

Related U.S. Application Data
(63) Continuation of application No. PCT/CN2024/128947, filed on Oct. 31, 2024.

(30) Foreign Application Priority Data
Dec. 25, 2023 (CN) 202311792309.4

(51) Int. Cl. A61K 39/12; A61K 9/127; A61K 9/51; A61K 39/00; A61P 31/14; C07K 14/005; C12N 7/00; C12N 15/66

(52) U.S. Cl. CPC A61K 39/12; A61K 9/127; A61K 9/5123; A61P 31/14; C07K 14/005; C12N 7/00; C12N 15/66; A61K 2039/5258; A61K 2039/55555; C12N 2760/00022; C12N 2760/00034; C12N 2830/50

(57) ABSTRACT

A messenger ribonucleic acid (mRNA) vaccine for Bandavirus dabieense (DBV) and a preparation method thereof are provided. The provided mRNA molecule is an mRNA obtained by cloning an optimized DBV glycoprotein gonadotropins (Gn) sequence into a pGEM-3Zf (+) mRNA vaccine vector, linearizing a plasmid via enzyme digestion, and capping and adding a poly(A) tail through an in vitro transcription enzyme method. The mRNA is encapsulated using a lipid nanoparticle delivery system to obtain the mRNA vaccine.

### CROSS-REFERENCE TO RELATED APPLICATIONS

This application is a continuation of International Patent Application No. PCT/CN2024/128947, filed Oct. 31, 2024 and claims priority of Chinese Patent Application No. 202311792309.4, filed on Dec. 25, 2023. The entire contents of International Patent Application No. PCT/CN2024/128947 and Chinese Patent Application No. 202311792309.4 are incorporated herein by reference

### INCORPORATION BY REFERENCE STATEMENT

This statement, made under Rules 77 (b) (5) (ii) and any other applicable rule incorporates into the present specification of an XML file for a “Sequence Listing XML” (see Rule 831 (a)), submitted via the USPTO patent electronic filing system or on one or more read-only optical discs (see Rule 1.52 (e) (8)), identifying the names of each file, the date of creation of each file, and the size of each file in bytes as follows:
File name: SequenceListing.xml
Creation date: Jul. 17, 2025
Byte size: 7,229

### TECHNICAL FIELD

The present disclosure relates to the field of biotechnology and pharmaceuticals, and in particular relates to a messenger ribonucleic acid (mRNA) vaccine for Bandavirus dabieense (DBV) and a preparation method thereof.

### BACKGROUND

Severe fever with thrombocytopenia syndrome (SFTS) is an emerging infectious disease caused by infection with the severe fever with thrombocytopenia syndrome virus (SFTSV). The disease is an acute infectious disease caused by a novel variant of Bunyavirus, and is named as SFTS, and the pathogen of SFTS is named as SFTSV. In 2019, the International Committee on Taxonomy of Viruses renamed the pathogen as Bandavirus dabieense (DBV). Severe patients may experience symptoms such as disseminated intravascular coagulation, multi-organ failure, persistent thrombocytopenia, and elevated inflammatory cytokine levels, and even death, with a mortality rate ranging from 12% to 30%. The World Health Organization (WHO) listed SFTS as a priority emerging infectious disease for research and intervention in 2017.

The DBV glycoprotein gonadotropins (Gn) binds to cell surface receptors, mediating viral entry into host cells. Additionally, the antigenic determinants contained in Gn may interact with host cells, induce the production of neutralizing antibodies against glycoproteins in the body, and activate cytotoxic T cells, inducing specific cellular immune responses. Specific antibodies targeting glycoprotein Gn may prevent the entry of pseudoviruses mediated by glycoprotein Gn into human embryonic kidney 293 cell line (HEK293) T cells, and this blocking effect is positively correlated with the titer of neutralizing antibodies. The above research suggests that DBV glycoprotein Gn is an important target molecule for inducing the production of neutralizing antibodies in the body.

Currently, there are no specific treatments or effective vaccines for SFTS. Vaccines play a vital role in infectious disease prevention. Messenger ribonucleic acid (mRNA) vaccines have made significant advancements in recent years. Compared to traditional vaccines, mRNA does not integrate into the genome, eliminating concerns about insertional mutations. The mRNA vaccines may be produced without cells, achieving rapid, economical, and efficient production. The mRNA vaccines may encode multiple antigens, enhancing immune responses against adaptable pathogens, and target multiple microbial or viral variants in a single formulation. Moreover, the development of effective vectors and the control of immunogenicity contribute to the successful application of mRNA vaccines in the prevention of infectious diseases, and the research and development technology of mRNA vaccines is maturing. The mRNA vaccines have emerged as a promising approach for preventing and treating various diseases.

The mRNA vaccines have been widely used in preventing and treating infectious diseases. Numerous preclinical and clinical trials of practical mRNA vaccines inducing antiviral immunity have been conducted in various infectious diseases, such as Zika virus, human immunodeficiency virus (HIV) virus, and influenza virus. The present disclosure is based on the mRNA vaccine strategy, designing and constructing an mRNA vaccine for DBV, evaluating the ability of the vaccine to transcribe in vitro and induce specific neutralizing antibodies in mice, which may provide candidate prevention strategies for the prevention and control of DBV.

### SUMMARY

The objective of the present disclosure is to leverage the advantages of messenger ribonucleic acid (mRNA) vaccines over inactivated vaccines, subunit vaccines, or deoxyribonucleic acid (DNA) vaccines, provide an mRNA molecule for Bandavirus dabieense (DBV), and confirm its expression of the DBV gonadotropins (Gn) protein through transfection into eukaryotic cells. Additionally, the prepared mRNA molecule is encapsulated with lipid nanoparticles (LNPs) to obtain an mRNA vaccine. The serum titer measurement and virus neutralization tests after immunization of mice confirm that the Gn mRNA vaccine may induce high levels of specific neutralizing antibodies in mice. Virus neutralization tests confirm that immune serum may bind to the virus and prevent virus infection of cells. The mRNA vaccine for DBV obtained by the present disclosure provides a candidate vaccine molecule for the prevention of DBV infection.

To address the above technical problems and achieve the objectives, the present disclosure provides the following technical solutions.

In one aspect, the present disclosure provides an mRNA vaccine for DBV, where a coding sequence of the mRNA is shown in SEQ ID No: 2.

Optionally, the mRNA is capped.

Optionally, the mRNA is an mRNA obtained by cloning an optimized DBV glycoprotein Gn sequence into a pGEM-3Zf (+) mRNA vaccine vector, linearizing a plasmid via enzyme digestion, and capping and adding a poly(A) tail through an in vitro transcription enzyme method.

The optimized DBV glycoprotein Gn sequence is shown in SEQ ID No: 2.

Optionally, the amino acid sequence of the DBV glycoprotein Gn is shown in SEQ ID No: 1.

In one aspect, the present disclosure provides an expression vector, where the expression vector expresses the aforementioned mRNA molecule.

Optionally, the expression vector is pGEM-3Zf (+).

In one aspect, the present disclosure provides an mRNA vaccine for DBV, where the mRNA is prepared using the aforementioned expression vector.

In one aspect, the present disclosure protects an application of any aforementioned mRNA vaccine or the aforementioned expression vector in preparing an mRNA vaccine composition for DBV.

In one aspect, the present disclosure protects a vaccine composition, where the vaccine composition includes any aforementioned mRNA vaccine or the aforementioned expression vector.

In one aspect, the present disclosure provides a preparation method of an mRNA vaccine for DBV, where firstly, a recombinant plasmid expressing the aforementioned mRNA vaccine for DBV is constructed, and an mRNA obtained by in vitro transcription is added with a Cap cap and a poly (A) tail, and then encapsulated with lipid nanoparticles to obtain the mRNA vaccine.

The present disclosure further provides a more specific preparation method of an mRNA vaccine for DBV, including following specific steps.

Design of mRNA Plasmid and Template Plasmid Preparation

The sequence information of glycoprotein Gn encoded by M fragment of DBV strain (JS2012-70) with GeneBank accession number: KY362350.1 is analyzed. After sequence optimization, the whole gene is synthesized and cloned into pGEM-3Zf (+) expression vector. The plasmid construction is verified for correctness by restriction endonuclease digestion and gene sequencing.

### MRNA Preparation and In Vitro Expression Verification

The correctly constructed plasmid is transformed into Escherichia coli for plasmid amplification. A plasmid midi preparation kit is used to extract and purify the plasmid from 200 milliliters (ml) of bacterial solution. The plasmid is linearized using BamH I restriction endonuclease, and the gel recovery kit is used for recovery. After capping and poly(A) tail addition, the prepared mRNA is transiently transfected into 293 FT cells using Lipofectamine transfection reagent and transfected for 24 hours. The expression of Gn is detected by western blot experiment.

Encapsulation of Messenger Ribonucleic Acid-Loaded Lipid Nanoparticle (mRNA-LNP)

The mRNA template preparation and lipid nanoparticle (LNP) encapsulation of the prepared mRNA supercoiled plasmid are completed.

Through the technical solutions, the present disclosure prepares the mRNA vaccine for DBV; the vaccine is used for immunization alone or sequential immunization with other types of Bunyavirus vaccines.

The present disclosure further protects the recombinant plasmid obtained by the aforementioned preparation method.

The present disclosure further protects the DBV vaccine obtained by the aforementioned preparation method.

### Beneficial Effects

According to the present disclosure, the Gn glycoprotein encoded by the M segment of DBV is selected as the immunogen, and the mRNA vaccine for DBV is successfully constructed. The mRNA vaccine may produce a high level of immune protection against Bunyavirus, including the DBV, demonstrating a high level of humoral immunity. Therefore, the vaccine has broad application prospects in the prevention of DBV infection.

### BRIEF DESCRIPTION OF THE DRAWINGS

One or more embodiments are illustrated by the corresponding figures in the accompanying drawings, and these exemplary illustrations do not constitute limitations on the embodiments. The term “exemplary” used here means “used as an example, implementation, or illustration”. Any embodiments illustrated here as “exemplary” need not be interpreted as superior or better than other embodiments. In order to provide a clearer explanation of the specific embodiments of the present disclosure or the technical solutions in the prior art, a brief introduction will be given to the accompanying drawings required for the description of the specific embodiments or the prior art. Apparently, that the accompanying drawings described below are some embodiments of the present disclosure. For one of ordinary skill in the art, other drawings may be obtained based on these drawings without creative labor.

FIG. 1 shows electrophoresis identification results of Bandavirus dabieense gonadotropins messenger ribonucleic acid (DBV Gn mRNA) plasmid.

FIG. 2A shows results of in vitro transcription identification for DBV Gn mRNA.

FIG. 2B shows results of in vitro transcription identification for DBV Gn mRNA.

FIG. 2C shows results of in vitro transcription identification for DBV Gn mRNA.

FIG. 3 shows expression of mRNA identified by Western Blot in 293 FT cells.

FIG. 4A shows expression levels of antibodies in mouse serum detected by enzyme-linked immunosorbent assay (ELISA) at 4 weeks.

FIG. 4B shows expression levels of antibodies in mouse serum detected by ELISA at 6 weeks.

FIG. 4C shows expression levels of antibodies in mouse serum detected by ELISA at 8 weeks.

FIG. 4D shows expression levels of antibodies in mouse serum detected by ELISA at 10 weeks.

FIG. 5 shows quantitative real-time polymerase chain reaction (qPCR) results of viral titers in serum neutralization tests.

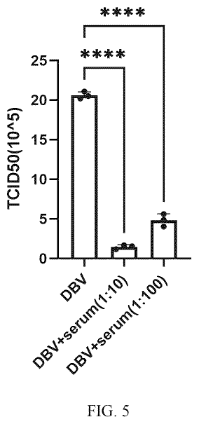

### DETAILED DESCRIPTION OF THE EMBODIMENTS

To clarify the objectives, technical solutions, and advantages of the embodiments of the present disclosure, the following describes the technical solutions of the embodiments in detail with reference to the accompanying drawings. It is evident that the described embodiments represent only a portion of the present disclosure, rather than all possible embodiments. The embodiments illustrated in the accompanying drawings may be designed and implemented in various different ways.

Therefore, the detailed description of the embodiments provided herein is not intended to limit the scope of the claimed present disclosure but merely to present selected embodiments. Based on the embodiments of the present disclosure, all other embodiments derived by those skilled in the art without creative effort shall fall within the protection scope of the present disclosure.

### Embodiment 1: Design of Messenger Ribonucleic Acid (mRNA) Plasmid and Template Plasmid Preparation

The amino acid sequence (as shown in SEQ ID No: 1) of the Bandavirus dabieense (DBV) strain (JS2012-70) with GeneBank accession number: KY362350.1 is analyzed, with a focus on the sequence information of glycoprotein gonadotropins (Gn) encoded by M fragment. After removing the signal peptide and transmembrane region, after sequence optimization, the whole gene (as shown in SEQ ID No: 2) is synthesized and cloned into pGEM-3Zf (+) expression vector. The plasmid construction is verified for correctness by restriction endonuclease digestion and gene sequencing.

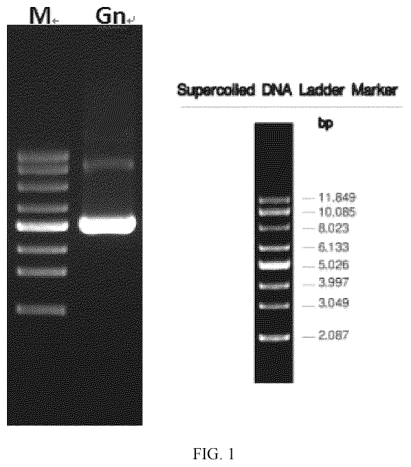

Through the sequence analysis of DBV Gn, cluster of differentiation 5 (CD5) signal peptide sequence is added, and the optimized gene sequence is cloned into the pGEM-3Zf (+) expression vector. Agarose gel electrophoresis identifies that the plasmid clone is correct, and the plasmid size is about 5172 base pairs (bp) (FIG. 1).

### Embodiment 2: MRNA Preparation and In Vitro Expression Validation

The correctly constructed plasmid is transformed into Escherichia coli for plasmid amplification. A plasmid midi preparation kit is used to extract and purify the plasmid from 200 milliliters (ml) of bacterial solution. The plasmid is linearized using BamH I restriction endonuclease, and the gel recovery kit is used for recovery. After capping and poly(A) tail addition, the prepared mRNA is transiently transfected into 293 FT cells using Lipofectamine transfection reagent and transfected for 24 hours (h). The expression of Gn is detected by western blot experiment.

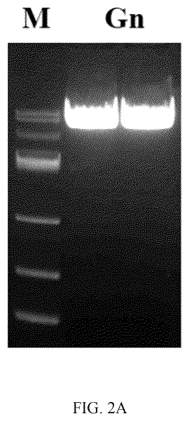

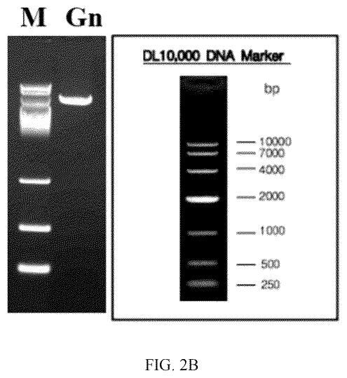

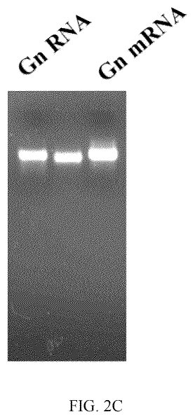

The successfully constructed mRNA plasmid template is linearized with BamH I enzyme digestion (FIG. 2A), and the linearized mRNA plasmid template is obtained by a gel extraction kit, with the concentration of 166.81 nanograms per microliter (ng/μl) (FIG. 2B). In vitro transcription is performed using T7 polymerase, followed by capping and poly(A) tail addition. Agarose gel electrophoresis detects the purity of ribonucleic acid (RNA) and mRNA, with in vitro transcription of 700 nanograms (ng) of RNA yielding 12.9 microgram (μg) of mRNA (FIG. 2C).

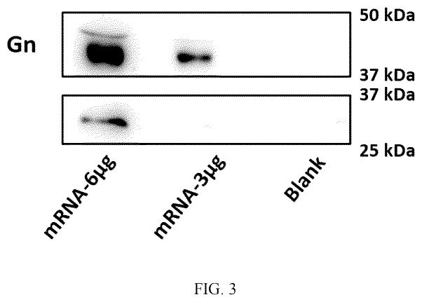

The obtained 3 μg and 6 μg mRNA are transfected into 293 FT cells using Lipofectamine 2000 and transfected for 6 h. The medium is replaced with fresh culture medium, and cells are collected after 24 h. The cells are lysed using radio immunoprecipitation assay (RIPA), and the expression of Gn is verified by western blot. The concentration of the primary antibody Gn is 1:1000, and the concentration of the secondary antibody goat anti-mouse is 1:5000. The results show that the prepared mRNA may be expressed in eukaryotic 293 FT cells (FIG. 3).

### Embodiment 3: Encapsulation of Messenger Ribonucleic Acid-Loaded Lipid Nanoparticle (mRNA-LNP)

The mRNA template preparation and lipid nanoparticle (LNP) encapsulation of the prepared mRNA supercoiled plasmid are completed. The encapsulation of mRNA-LNP is entrusted to Novoprotein Scientific (Shanghai) Inc.

After detection, the volume of DBV-Gn mRNA-LNP prepared by the present disclosure in this batch is 1.62 ml; the mass is 0.40 milligram (mg); the appearance (eye inspection) is transparent or ivory suspension; the encapsulation efficiency (fluorescence method) is 97.70%; the mRNA concentration (fluorescence method) is 247.15 ng/μl; the particle size (dynamic light scattering) is 84.65 nanometers (nm), and the polydispersity index (dynamic light scattering) is 0.160; the Zeta potential (pulse amplitude locked system (PALS) Zeta potential) measurement is −0.60 millivolt (mV); and pH value (pH meter) is 7.43.

### Embodiment 4: Immunization of BALB/c Mice with mRNA-LNP

Mice are immunized according to the following immunization scheme. Female BALB/c mice aged 6-8 weeks are divided into low, medium, and high immunization dose groups (n=5 in each group), with injection doses of 2 μg/mouse, 5 μg/mouse, and 20 μg/mouse, respectively. Immunization is administered intramuscularly every 2 weeks. Four weeks later, mouse serum is collected for enzyme-linked immunosorbent assay (ELISA) analysis to evaluate the neutralizing antibody titers induced by mRNA vaccine in mice.

### Embodiment 5: ELISA detection of mouse serum antibody expression

The Gn glycoprotein expressed in eukaryotic cells is encapsulated in ELISA plates at 100 nanograms per well (ng/well). Blood is collected from the tail vein of mice, and serum is collected, and the serum is diluted in gradient with the initial concentration of 1:100, and each dilution titer is set with three multiple holes. The absorbance of OD_{450 nm} is 2.1 times that of the blank control, and the titer level of mouse serum antibodies is interpreted.

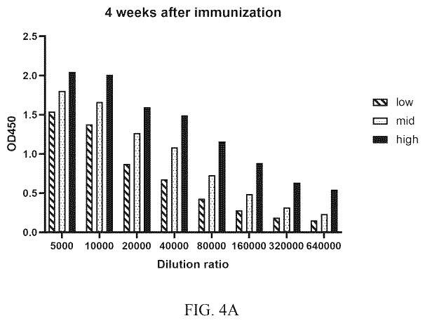

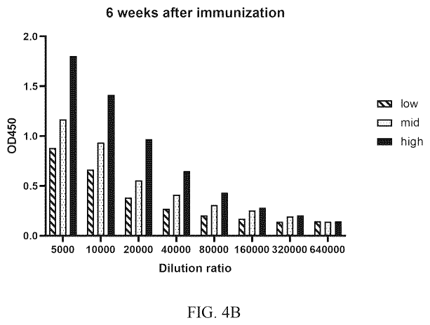

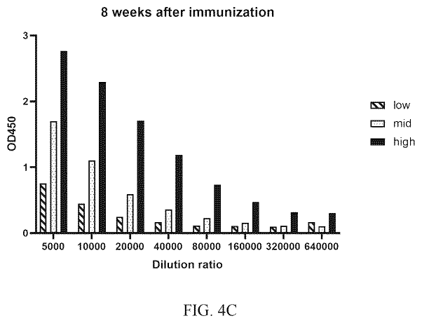

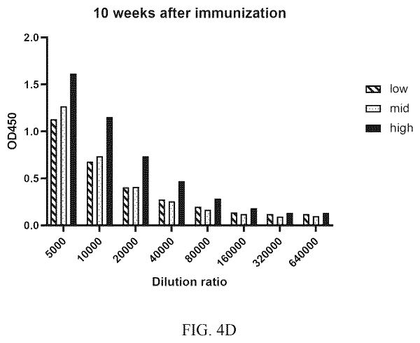

Mice are immunized as planned, and mouse serum is collected from the 4th week, and mouse serum titers are continuously measured at 6th week, 8th week and 10th week. The results show that the antibody titers of the three immunization dose groups all reach 1:640,000 after 4 weeks of immunization (FIG. 4A), and the antibody titer level gradually decreases as time goes on. By the 10th week of immunization, the antibody titers of the high-dose immunization group may still reach 1:160,000 (FIG. 4A-FIG. 4D).

### Embodiment 6: Virus Neutralization Assay

Vero cells are inoculated into a 6-well plate, and when the cell confluence reaches 90%, the virus is inoculated. The virus and mouse serum are diluted separately with dulbecco's modified eagle medium (DMEM) cell culture medium (dilution ratios of 1:10, 1:100). The virus inoculation dose is multiplicity of infection (MOI)=0.01. 500 microliter (μl) virus solution is mixed with 500 μl serum diluent and incubated at 37° C. for 30 min. At the same time, a serum-free blank control is set up. The medium in the 6-well plate is removed, and 1 ml of mixed solution of virus and serum is added, and 3 parallel wells are set at each dilution. After incubation at 37° C. for 2 h, the mixed solution is removed and 2 ml of cell maintenance solution is added. After 24 h, the virus titer is measured according to the instructions of Bunyavirus nucleic acid detection kit (DaAn Gene, DA0340) for severe fever with thrombocytopenia syndrome.

The DB virus is incubated with mouse serum at different dilution ratios (1:10 and 1:100) at MOI-0.01, and then inoculated into Vero cells. The virus titer is measured by quantitative real-time polymerase chain reaction (qPCR) after 24 h of infection. The results show that the serum of immunized mice can prevent virus infection of cells, indicating that mRNA vaccine immunization can induce specific neutralization of mouse expression.

What is claimed is:

1. A messenger ribonucleic acid (mRNA) vaccine for Bandavirus dabieense, wherein a coding sequence of the mRNA is shown in SEQ ID No: 2.

2. The mRNA vaccine according to claim 1, wherein the mRNA is capped.

3. The mRNA vaccine according to claim 2, wherein the mRNA is an mRNA obtained by cloning a sequence shown in SEQ ID No: 2 into a pGEM-3Zf (+) mRNA vaccine vector, linearizing a plasmid via enzyme digestion, and capping and adding a poly(A) tail through an in vitro transcription enzyme method.

4. The mRNA vaccine according to claim 1, wherein an amino acid sequence of Bandavirus dabieense glycoprotein Gn is shown in SEQ ID No: 1.

5. An expression vector, wherein the expression vector expresses a molecule of the mRNA according to claim 1.

6. A preparation method of a messenger ribonucleic acid (mRNA) vaccine for Bandavirus dabieense, wherein firstly, a recombinant plasmid expressing the mRNA vaccine for Bandavirus dabieense according to claim 1 is constructed, and an mRNA obtained by in vitro transcription is added with a Cap cap and a poly(A) tail, and then encapsulated with lipid nanoparticles to obtain the mRNA vaccine.
